(19) United States
(12) Patent Application Publication
(10) Pub. No.: US 2002/0013520 A1
(43) Pub. Date: Jan. 31, 2002

(54) System, method, database server and record medium for at-home health support

(75) Inventor: Katsuro Okamoto, Tokyo (JP)

Correspondence Address: OSTROLENK FABER GERB & SOFFEN, 1180 AVENUE OF THE AMERICAS, NEW YORK, NY, 100368403

(73) Assignee: NEC Corporation

(21) Appl. No.: 09/906,543

(22) Filed: Jul. 16, 2001

(30) Foreign Application Priority Data
Jul. 17, 2000 (JP) 216426/2000

(51) Int. Cl. A61B 5/00

(52) U.S. Cl. 600/300; 705/3

(57) ABSTRACT

A health care database of an at-home health support system operated by a municipality for residents of the municipality is constructed in a server of a company that is connected to the Internet, and the municipality commissions the company to manage the health care database, thereby cost for the at-home health support system is reduced. Vital data inputted by a resident are transmitted from a resident's health care terminal unit to the server via the Internet and are registered in the health care database. The municipality using a municipality's terminal unit refers to the vital data in the health care database via the Internet, makes a diagnosis/advice for the vital data, and registers the diagnosis/advice in the health care database via the Internet. When the diagnosis/advice can not be made by the municipality, commission information is sent to a terminal unit of an outside medical institution via the Internet, and the outside medical institution makes the diagnosis/advice and registers the diagnosis/advice in the health care database via the Internet. Thereafter, the resident using the resident's health care terminal unit makes access to the server via the Internet and refers to the diagnosis/advice which has been registered in the health care database. Database management cost calculated by the server and diagnosis cost calculated by the outside medical institution's terminal unit are charged to the municipality via the Internet.

### BACKGROUND OF THE INVENTION

The present invention relates to a system, method, database server and record medium for at-home health support, and in particular, to a system, method, database server and record medium for at-home health support which is operated for residents of a municipality under a commission from the municipality to a company having a server that is connected to the Internet.

Description of the Related Art

In recent years, people are getting more and more health conscious because of the spread of lifestyle-related diseases etc. and there is an increasing need for appropriate health guidance, thereby at-home health support systems for managing health conditions of residents in everyday life at home by giving them health guidance without taking them out to medical institutions etc. are being introduced into municipalities etc. In a typical conventional at-home health support system which is operated by a municipality, each resident sends his/her vital data (blood pressure, electrocardiogram, etc.) to the municipality every day, and the municipality makes a diagnosis and/or advice for the vital data and sends the diagnosis/advice to the resident. For the implementation of the at-home health support system, health care terminal units are generally provided to houses of the residents and a health care database server of the municipality is connected to the health care terminal units via a network. Therefore, the health care database server of the municipality is required to have databases of large capacities, and the construction, operation and management (update, maintenance, etc.) of the databases become important tasks of the municipality.

As described above, a municipality that intends to operate an at-home health support system has to have its own large-capacity health care database server and has to construct, operate and manage (update, maintain, etc.) the databases of the health care database server by itself. Therefore, considerable amounts of capital investment, time and trouble are required of the municipality for the operation of the at-home health support system, regardless of whether the number of the users of the at-home health support system is large or small.

Further, individuals living out of the municipality could not make access to the health care database server of the municipality easily since cost for exclusive lines and special-purpose software for making access to the health care database server were necessary.

### SUMMARY OF THE INVENTION

It is therefore the primary object of the present invention to provide a system, a method, a database server and a record medium for at-home health support which is operated for residents of a municipality, by which cost, time and trouble required for the operation of the at-home health support system can be reduced.

Another object of the present invention is to provide a system, a method, a database server and a record medium for at-home health support which is operated for residents of a municipality, by which individuals living out of the municipality such as relatives of the residents are allowed to make access to the health care database server easily.

In accordance with a first aspect of the present invention, there is provided an at-home health support system in which a health care database for residents of a municipality is constructed in a server (3) of a company that is connected to the Internet (6) and the municipality commissions the company to manage the health care database. The at-home health support system comprises vital information registration means (11), a database reference means (21), a first diagnosis/advice registration means (22), a diagnosis/advice commissioning means (23), a second diagnosis/advice registration means (42) and diagnosis/advice reference means (12). The vital information registration means (11) are provided to the residents for sending vital data and/or medical questionnaire data of the resident to the server (3) via the Internet (6) and thereby registering the data of the resident in the health care database of the server (3). The database reference means (21) is provided to the municipality for making access to the server (3) via the Internet (6) and thereby referring to the vital data and/or medical questionnaire data of the residents which have been registered in the health care database of the server (3). The first diagnosis/advice registration means (22) is provided to the municipality for sending a diagnosis and/or advice made by the municipality for the vital data and/or medical questionnaire data of a resident to the server (3) via the Internet (6) and thereby registering the diagnosis/advice for the resident in the health care database of the server (3). The diagnosis/advice commissioning means (23) is provided to the municipality for making access to a terminal unit (4) of an outside medical institution or health service company via the Internet (6) and thereby commissioning the outside medical institution or health service company to make a diagnosis and/or advice for the vital data and/or medical questionnaire data of a resident when the diagnosis/advice for the resident can not be made by the municipality. The second diagnosis/advice registration means (42) is provided to the outside medical institution or health service company for sending a diagnosis and/or advice made by the outside medical institution or health service company under the commission from the municipality for the vital data and/or medical questionnaire data of the resident to the server (3) via the Internet (6) and thereby registering the diagnosis/advice for the resident in the health care database of the server (3). The diagnosis/advice reference means (12) are provided to each resident for making access to the server (3) via the Internet (6) and thereby referring to the diagnosis/advice which has been registered in the health care database of the server (3).

In accordance with a second aspect of the present invention, in the first aspect, the at-home health support system further comprises a management cost calculation charging means (34) and a diagnosis cost calculation charging means (43). The management cost calculation charging means (34) is provided to the server (3) for calculating management cost of the health care database and charging the municipality for the management cost via the Internet (6). The diagnosis cost calculation charging means (43) is provided to the outside medical institution or health service company for calculating diagnosis cost for the diagnoses/advice made by the outside medical institution or health service company under the commission from the municipality and charging the municipality for the diagnosis cost via the Internet (6).

In accordance with a third aspect of the present invention, in the first aspect, the server (3) includes a user verification means (31) for checking whether or not each user making access to the server (3) via the Internet (6) is a registered user.

In accordance with a fourth aspect of the present invention, in the first aspect, the at-home health support system further comprises second diagnosis/advice reference means (51). The second diagnosis/advice reference means (51) are provided to registered specific users located outside the municipality for making access to the server (3) via the Internet (6) and thereby letting the specific user refer to the diagnosis/advice which has been registered in the health care database of the server (3) for a resident related to the specific user.

In accordance with a fifth aspect of the present invention, in the fourth aspect, the specific users include relatives of residents of the municipality.

In accordance with a sixth aspect of the present invention, in the first aspect, the company to which the municipality commissions the management of the health care database is an ISP (Internet Service Provider).

In accordance with a seventh aspect of the present invention, there is provided an at-home health support system in which a health care database for residents of a municipality is constructed in a server (3) of a company that is connected to the Internet (6) and the municipality commissions the company to manage the health care database. The at-home health support system comprises: the server (3); resident's health care terminal units (1) which are provided to residents of the municipality for making access to the Internet (6); a municipality's terminal unit (2) which is provided to the municipality for making access to the Internet (6); and a medical institution/health service company's terminal unit (4) which is provided to an outside medical institution or health service company for making access to the Internet (6). The resident's health care terminal unit (1) includes: a vital information input transmission means (11) for letting a resident input his/her vital data and/or medical questionnaire data and transmitting the data to the server (3) via the Internet (6) together with a user ID and a password so that the data of the resident will be registered in the health care database of the server (3); and a result reference means (12) for making access to the server (3) via the Internet (6) and thereby referring to a diagnosis/advice which has been made for the vital data and/or medical questionnaire data and registered in the health care database of the server (3). The municipality's terminal unit (2) includes: a database reference means (21) for making access to the server (3) via the Internet (6) and thereby referring to the vital data and/or medical questionnaire data of the residents which have been registered in the health care database of the server (3); a first diagnosis/advice input transmission means (22) for letting the municipality input a diagnosis and/or advice for the vital data and/or medical questionnaire data of a resident and transmitting the diagnosis/advice to the server (3) via the Internet (6) so that the diagnosis/advice for the resident will be registered in the health care database of the server (3); and a commission information generation transmission means (23) for generating commission information when the diagnosis/advice for a resident is commissioned to an outside medical institution or health service company and transmitting the commission information to a terminal unit (4) of the outside medical institution or health service company via the Internet (6). The server (3) includes: a user information database (35) in which resident information of each resident such as name, user ID, password, date of birth and sex and password information concerning the correspondence between each password and data/database that can be referred to by use of the password are registered and stored; a vital information database (36) in which the vital data, medical questionnaire data, etc. of each resident are registered and stored; an information provision database (37) in which the diagnosis, advice, etc. for each resident are registered and stored; a user information registration verification means (31) for registering user information such as the resident information and the password information in the user information database (35) and checking whether or not each user making access to the server (3) via the Internet (6) is a registered user by use of the user information registered in the user information database (35); a vital information registration means (32) for registering the vital data and/or medical questionnaire data supplied from the resident's health care terminal units (1) in the vital information database (36) with regard to each resident; and a diagnosis/advice registration means (33) for registering the diagnosis/advice supplied from the municipality's terminal unit (2) or the medical institution/health service company's terminal unit (4) in the information provision database (37) with regard to each resident. The medical institution/health service company's terminal unit (4) includes: a database reference means (41) for making access to the server (3) via the Internet (6) and thereby referring to the vital data and/or medical questionnaire data of the residents which have been registered in the health care database of the server (3); and a second diagnosis/advice input transmission means (42) for letting the outside medical institution or health service company input a diagnosis and/or advice for the vital data and/or medical questionnaire data of a resident and transmitting the diagnosis/advice to the server (3) via the Internet (6) so that the diagnosis/advice for the resident will be registered in the health care database of the server (3).

In accordance with an eighth aspect of the present invention, in the seventh aspect, the server (3) further includes a management cost calculation charging means (34) for calculating management cost of the health care database and charging the municipality for the management cost by sending a bill to the municipality's terminal unit (2) via the Internet (6). The medical institution/health service company's terminal unit (4) further includes a diagnosis cost calculation charging means (43) for calculating diagnosis cost for the diagnoses/advice made by the outside medical institution or health service company under the commission from the municipality and charging the municipality for the diagnosis cost by sending a bill to the municipality's terminal unit (2) via the Internet (6).

In accordance with a ninth aspect of the present invention, in the seventh aspect, the at-home health support system further comprises specific user's terminal units (5). The specific user's terminal units (5) are provided to registered specific users located outside the municipality for making access to the server (3) via the Internet (6) and thereby letting the specific user refer to the diagnosis/advice which has been registered in the health care database of the server (3) with respect to a resident related to the specific user.

In accordance with a tenth aspect of the present invention, in the ninth aspect, the specific users include relatives of residents of the municipality.

In accordance with an eleventh aspect of the present invention, in the ninth aspect, the user information registration verification means (31) of the server (3) also registers information concerning the correspondence between user IDs of the residents and passwords of the registered specific users in the user information database (35) so that each registered specific user using a password will be allowed to refer to data of one or more residents having user IDs corresponding to the password.

In accordance with a twelfth aspect of the present invention, in the seventh aspect, the server (3) further includes a health-related information database (38) which preliminarily stores a variety of prepared contents concerning diet regimens, exercise regimens, rehabilitation programs, etc. When the diagnosis/advice is transmitted by the first diagnosis/advice input transmission means (22) of the municipality's terminal unit (2) or the second diagnosis/advice input transmission means (42) of the medical institution/health service company's terminal unit (4) to the server (3), health-related information related to the diagnosis/advice is obtained from the health-related information database (38) and the health-related information is transmitted to the server (3) together with the diagnosis/advice. The diagnosis/advice registration means (33) of the server (3) registers the health-related information in the information provision database (37) together with the diagnosis/advice. The result reference means (12) of the resident's health care terminal unit (1) refers to the health-related information together with the diagnosis/advice.

In accordance with a thirteenth aspect of the present invention, in the seventh aspect, the company to which the municipality commissions the management of the health care database is an ISP (Internet Service Provider).

In accordance with a fourteenth aspect of the present invention, there is provided a health care database server (3) of a system including a municipality's terminal unit (2) connected to the Internet (6), resident's health care terminal units (1) connected to the Internet (6), a medical institution/health service company's terminal unit (4) connected to the Internet (6), and the health care database server (3) connected to the Internet (6) and owned by a company to which the municipality commissions the management of the health care database server (3). The health care database server (3) comprises a user information database (35), a vital information database (36), an information provision database (37), a user information registration verification means (31), a vital information registration means (32) and a diagnosis/advice registration means (33). In the user information database (35), resident information of each resident of the municipality such as name, user ID, password, date of birth and sex and password information concerning the correspondence between each password and data/database that can be referred to by use of the password are registered and stored. In the vital information database (36), vital data, medical questionnaire data, etc. of each resident which are sent from the resident's health care terminal units (1) are registered and stored. In the information provision database (37), diagnosis, advice, etc. for each resident which are sent from the municipality's terminal unit (2) or the medical institution/health service company's terminal unit (4) are registered and stored. The user information registration verification means (31) registers user information such as the resident information and the password information in the user information database (35), and checks whether or not each user making access to the health care database server (3) via the Internet (6) is a registered user by use of the user information registered in the user information database (35) when access from the municipality's terminal unit (2), the resident's health care terminal unit (1) or the medical institution/health service company's terminal unit (4) occurred. The vital information registration means (32) registers the vital data and/or medical questionnaire data supplied from the resident's health care terminal units (1) via the Internet (6) in the vital information database (36) with regard to each resident so as to be referred to by the municipality's terminal unit (2) or the medical institution/health service company's terminal unit (4). The diagnosis/advice registration means (33) registers the diagnosis/advice supplied from the municipality's terminal unit (2) or the medical institution/health service company's terminal unit (4) via the Internet (6) in the information provision database (37) with regard to each resident so as to be referred to by the resident's health care terminal unit (1).

In accordance with a fifteenth aspect of the present invention, in the fourteenth aspect, the health care database server (3) further comprises a management cost calculation charging means (34) for calculating management cost of the health care database server (3) and charging the municipality for the management cost by sending a bill to the municipality's terminal unit (2) via the Internet (6).

In accordance with a sixteenth aspect of the present invention, there is provided an at-home health support method in which a health care database for residents of a municipality is constructed in a server (3) of a company that is connected to the Internet (6) and the municipality commissions the company to manage the health care database. The at-home health support method comprises a vital information registration step, a database reference step, a first diagnosis/advice registration step, a diagnosis/advice commissioning step, a second diagnosis/advice registration step and a diagnosis/advice reference step. In the vital information registration step, a resident sends his/her vital data and/or medical questionnaire data to the server (3) via the Internet (6) by use of a resident's health care terminal unit (1) and thereby the data of the resident is registered in the health care database of the server (3). In the database reference step, the municipality makes access to the server (3) via the Internet (6) by use of a municipality's terminal unit (2) and thereby refers to the vital data and/or medical questionnaire data of the resident which have been registered in the health care database of the server (3). In the first diagnosis/advice registration step, the municipality makes a diagnosis and/or advice for the vital data and/or medical questionnaire data of the resident if possible and sends the diagnosis/advice from the municipality's terminal unit (2) to the server (3) via the Internet (6) and thereby the diagnosis/advice for the resident is registered in the health care database of the server (3). In the diagnosis/advice commissioning step, the municipality makes access to a medical institution/health service company's terminal unit (4) of an outside medical institution or health service company via the Internet (6) by use of the municipality's terminal unit (2) and thereby commissions the outside medical institution or health service company to make a diagnosis and/or advice for the vital data and/or medical questionnaire data of the resident if the diagnosis/advice for the resident should be commissioned to the outside medical institution or health service company. In the second diagnosis/advice registration step, the outside medical institution or health service company makes a diagnosis and/or advice for the vital data and/or medical questionnaire data of the resident under the commission from the municipality and sends the diagnosis/advice from the medical institution/health service company's terminal unit (4) to the server (3) via the Internet (6) and thereby the diagnosis/advice for the resident is registered in the health care database of the server (3). In the diagnosis/advice reference step, the resident makes access to the server (3) via the Internet (6) by use of the resident's health care terminal unit (1) and thereby refers to the diagnosis/advice which has been registered in the health care database of the server (3).

In accordance with a seventeenth aspect of the present invention, in the sixteenth aspect, the at-home health support method further comprises a management cost calculation charging step and a diagnosis cost calculation charging step. In the management cost calculation charging step, management cost of the health care database is calculated by the server (3) and the management cost is charged to the municipality by sending a bill to the municipality's terminal unit (2) via the Internet (6). In the diagnosis cost calculation charging step, diagnosis cost for the diagnoses/advice made by the outside medical institution or health service company under the commission from the municipality is calculated by the medical institution/health service company's terminal unit (4) and the diagnosis cost is charged to the municipality by sending a bill to the municipality's terminal unit (2) via the Internet (6).

In accordance with an eighteenth aspect of the present invention, in the sixteenth aspect, the outside medical institution or health service company which received the commission from the municipality in the diagnosis/advice commissioning step makes access to the server (3) via the Internet (6) by use of the medical institution/health service company's terminal unit (4) and thereby refers to the vital data and/or medical questionnaire data of the resident which have been registered in the health care database of the server (3).

In accordance with a nineteenth aspect of the present invention, in the sixteenth aspect, the at-home health support method further comprises a user verification step. In the user verification step, the authenticity of a user making access to the server (3) via the Internet (6) as a registered user is checked.

In accordance with a twentieth aspect of the present invention, in the sixteenth aspect, the at-home health support method further comprises a second diagnosis/advice reference step. In the second diagnosis/advice reference step, a registered specific user located outside the municipality makes access to the server (3) via the Internet (6) by use of a specific user's terminal unit (5) and thereby refers to the diagnosis/advice which has been registered in the health care database of the server (3) for a resident related to the specific user.

In accordance with a twenty-first aspect of the present invention, in the twentieth aspect, the specific users include relatives of residents of the municipality.

In accordance with a twenty-second aspect of the present invention, in the sixteenth aspect, the company to which the municipality commissions the management of the health care database is an ISP (Internet Service Provider).

In accordance with a twenty-third aspect of the present invention, there is provided a computer-readable record medium storing a program for instructing a computer etc. to operate as a health care database server (3) of a system including a municipality's terminal unit (2) connected to the Internet (6), resident's health care terminal units (1) connected to the Internet (6), a medical institution/health service company's terminal unit (4) connected to the Internet (6), and the health care database server (3) connected to the Internet (6) and owned by a company to which the municipality commissions the management of the health care database server (3). The health care database server (3) implemented by the computer etc. and the program comprises a user information database (35), a vital information database (36), an information provision database (37), a user information registration verification means (31), a vital information registration means (32) and a diagnosis/advice registration means (33). In the user information database (35), resident information of each resident of the municipality such as name, user ID, password, date of birth and sex and password information concerning the correspondence between each password and data/database that can be referred to by use of the password are registered and stored. In the vital information database (36), vital data, medical questionnaire data, etc. of each resident which are sent from the resident's health care terminal units (1) are registered and stored. In the information provision database (37), diagnosis, advice, etc. for each resident which are sent from the municipality's terminal unit (2) or the medical institution/health service company's terminal unit (4) are registered and stored. The user information registration verification means (31) registers user information such as the resident information and the password information in the user information database (35), and checks whether or not each user making access to the health care database server (3) via the Internet (6) is a registered user by use of the user information registered in the user information database (35) when access from the municipality's terminal unit (2), the resident's health care terminal unit (1) or the medical institution/health service company's terminal unit (4) occurred. The vital information registration means (32) registers the vital data and/or medical questionnaire data supplied from the resident's health care terminal units (1) via the Internet (6) in the vital information database (36) with regard to each resident so as to be referred to by the municipality's terminal unit (2) or the medical institution/health service company's terminal unit (4). The diagnosis/advice registration means (33) registers the diagnosis/advice supplied from the municipality's terminal unit (2) or the medical institution/health service company's terminal unit (4) via the Internet (6) in the information provision database (37) with regard to each resident so as to be referred to by the resident's health care terminal unit (1).

In accordance with a twenty-fourth aspect of the present invention, in the twenty-third aspect, the health care database server (3) implemented by the computer etc. and the program further comprises a management cost calculation charging means (34) for calculating management cost of the health care database server (3) and charging the municipality for the management cost by sending a bill to the municipality's terminal unit (2) via the Internet (6).

### BRIEF DESCRIPTION OF THE DRAWINGS

The objects and features of the present invention will become more apparent from the consideration of the following detailed description taken in conjunction with the accompanying drawings, in which:

FIG. 1 is a block diagram showing an at-home health support system in accordance with an embodiment of the present invention;

FIG. 2 is a flow chart showing an example of the operation of the at-home health support system of FIG. 1; and

FIG. 3 is a block diagram showing an example of the implementation of the health care database server 3 of the at-home health support system of FIG. 1.

### DESCRIPTION OF THE PREFERRED EMBODIMENTS

Referring now to the drawings, a description will be given in detail of preferred embodiments in accordance with the present invention.

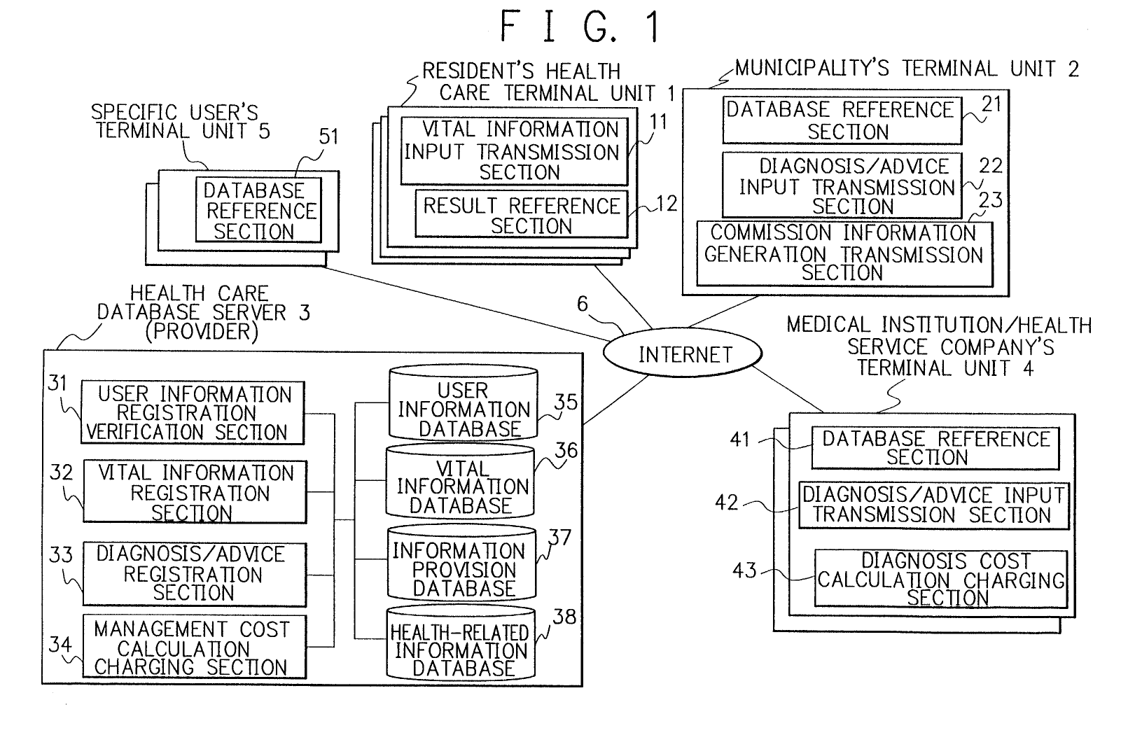

FIG. 1 is a block diagram showing an at-home health support system in accordance with an embodiment of the present invention. The at-home health support system shown in FIG. 1 includes resident's health care terminal units 1, a municipality's terminal unit 2, a health care database server 3, medical institution/health service company's terminal units 4, and specific user's terminal units 5. The terminal units 1, 2, 4 and 5 and the health care database server 3 are capable of making access to the Internet 6. Each terminal unit (1, 2, 4, 5 ) is implemented by, for example, a personal computer, a special-purpose computer, a PDA (personal digital assistant) or a cellular phone.

The resident's health care terminal unit 1, which is provided to houses of residents, public halls, community centers, meeting places, care facilities, etc., includes a vital information input transmission section 11 and a result reference section 12. The resident operates the vital information input transmission section 11 of the resident's health care terminal unit 1 and thereby inputs vital data (blood pressure, electrocardiogram, pulse rate, body temperature, blood-sugar level, body weight, etc.), medical questionnaire data (answers of the user to a medical questionnaire), a password, a user ID, etc. The information inputted to the vital information input transmission section 11 is transmitted to the health care database server 3 via the Internet 6. The result reference section 12 lets the user refer to a diagnosis, advice, etc. made for the vital data and the medical questionnaire data inputted by the resident.

The municipality's terminal unit 2, which is provided to a municipality, includes a database reference section 21, a diagnosis/advice input transmission section 22 and a commission information generation transmission section 23.

The database reference section 21 makes access to the health care database server 3 via the Internet 6 and thereby refers to data (vital data, medical questionnaire data, etc.) which have been stored in databases of the health care database server 3. The staff (medical team etc.) of the municipality (hereafter, simply referred to as “the municipality”) makes a diagnosis and/or advice for the referred vital data and/or medical questionnaire data of a resident and inputs the diagnosis/advice to the diagnosis/advice input transmission section 22. The diagnosis/advice input transmission section 22 sends the diagnosis/advice to the health care database server 3 via the Internet 6. The commission information generation transmission section 23 generates and transmits commission information (user ID of the resident, for example) to a medical institution/health service company's terminal unit 4 when the municipality commissions an outside medical institution or health service company to make a diagnosis/advice instead of the municipality.

The health care database server 3 is a server which is owned and managed by a company such as a provider (ISP (Internet Service Provider) etc.). The company is commissioned by the municipality to construct, operate and manage (update, maintain, etc.) databases for the health care of residents of the municipality. The health care database server 3 includes a user information registration verification section 31, a vital information registration section 32, a diagnosis/advice registration section 33, a management cost calculation charging section 34, a user information database 35, a vital information database 36, an information provision database 37 and a health-related information database 38.

The user information registration verification section 31 is used for registering “resident information” (name, date of birth, sex, age, user ID, password, etc.) of residents of the municipality in the user information database 35 under a commission from the municipality. In the user information database 35, information concerning the correspondence between user IDs of the residents and passwords of the “specific users” (that is, users of the specific user's terminal units 5) is also registered and stored by the user information registration verification section 31 to be used for verifying each “specific user” and thereby determining which data (that is, data of which resident) can be referred to by the “specific user”. Therefore, when a “specific user” using the specific user's terminal unit 5 and inputting a password makes access to the health care database server 3, the “specific user” is allowed to refer to data of one or more residents having user IDs corresponding to the password. For the municipality, medical institutions and health service companies, a special password (allowing access to all the data stored in the databases of the health care database server 3) is prepared, and the special password (corresponding to all the user IDs) is registered and stored in the user information database 35.

The vital information registration section 32 receives the vital data and/or medical questionnaire data from the resident's health care terminal units 1 via the Internet 6, and registers and stores the vital data and/or medical questionnaire data in the vital information database 36 with regard to each resident.

The diagnosis/advice registration section 33 receives the diagnoses/advice for residents from the municipality's terminal unit 2 and the medical institution/health service company's terminal units 4 via the Internet 6, and registers and stores the diagnoses/advice in the information provision database 37 with regard to each resident.

The management cost calculation charging section 34 calculates management cost of the databases periodically (once a month, for example) and charges the municipality for the management cost by sending a bill to the municipality's terminal unit 2 via the Internet 6.

The user information database 35 stores the aforementioned resident information which has been registered by the user information registration verification section 31. The vital information database 36 stores the vital data and the medical questionnaire data with regard to each resident. The information provision database 37 stores the diagnoses, advice and health-related information (which is extracted from the health-related information database 38), with regard to each resident. In the health-related information database 38, a variety of prepared contents concerning diet regimens, exercise regimens, rehabilitation programs, etc. are preliminarily stored.

The medical institution/health service company's terminal unit 4, which is provided to medical institutions or health service companies for giving diagnoses and/or advice to the residents under a commission from the municipality, includes a database reference section 41, a diagnosis/advice input transmission section 42 and a diagnosis cost calculation charging section 43.

The database reference section 41 makes access to the health care database server 3 via the Internet 6 and thereby refers to data (vital data, medical questionnaire data, etc.) which have been stored in the databases of the health care database server 3. The staff of the medical institution or health service company (hereafter, simply referred to as “the medical institution or health service company”) makes a diagnosis and/or advice for the referred vital data and/or medical questionnaire data of a resident and inputs the diagnosis/advice to the diagnosis/advice input transmission section 42. The diagnosis/advice input transmission section 42 sends the diagnosis/advice to the health care database server 3 via the Internet 6. The diagnosis cost calculation charging section 43 calculates diagnosis cost for the residents on each diagnosis or periodically (once a month, for example) and charges the municipality for the diagnosis cost by sending a bill to the municipality's terminal unit 2 via the Internet 6.

The specific user's terminal unit 5, which is provided to the house etc. of the aforementioned “specific user”, includes a database reference section 51. The “specific users” mean individuals who have been registered as registered users of the health care database server 3. The “specific users” can include individuals located outside the municipality such as relatives of residents of the municipality. The specific user's terminal units 5 are effectively used by specific users living outside the municipality. Such a specific user is allowed to make access to the databases of the health care database server 3 via the Internet 6 by use of the database reference section 51 and thereby refer to health conditions etc. of the resident who is related to the specific user.

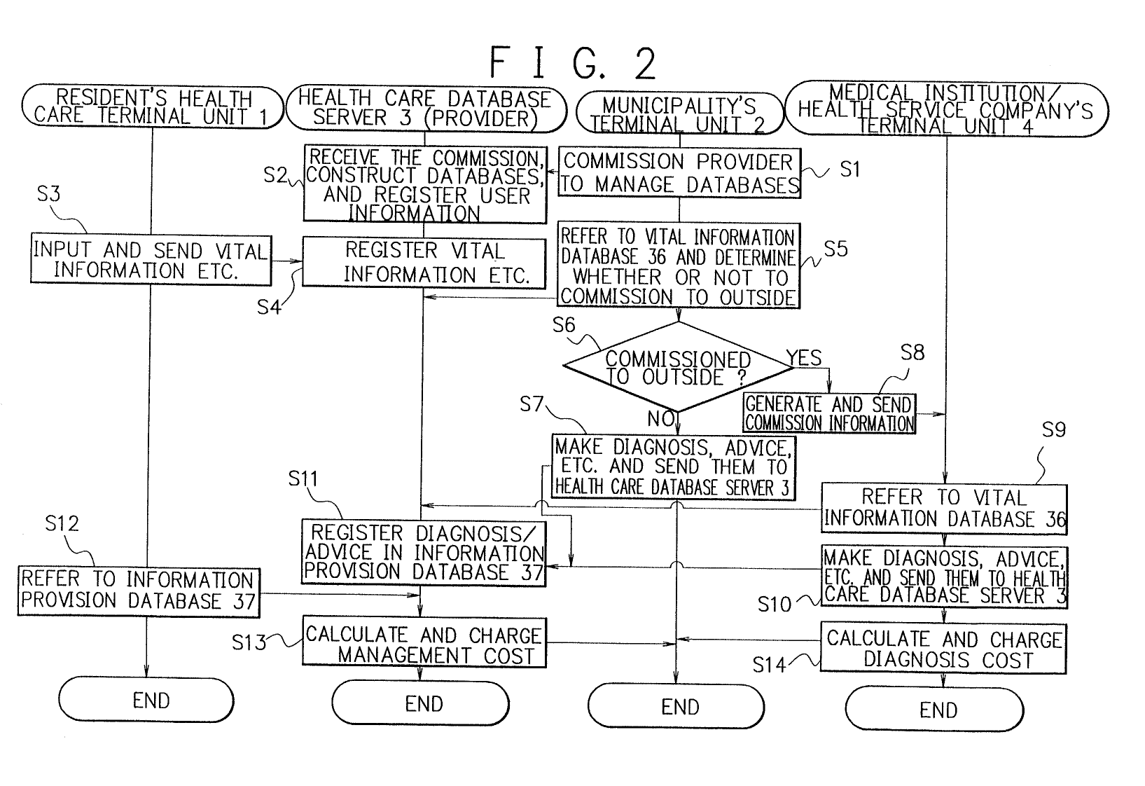

In the following, the operation of the at-home health support system in accordance with the embodiment of the present invention will be described in detail referring to FIG. 1 and FIG. 2. FIG. 2 is a flow chart showing an example of the operation of the at-home health support system of FIG. 1.

First, a municipality commissions a provider (ISP etc.) to construct, operate and manage (update, maintain, etc.) databases for the health care of residents of the municipality (step S1). The provider which received the commission from the municipality conducts the construction of the databases etc. of the health care database server 3, the information registration of users of the health care database server 3, etc. (step S2).

Each resident of the municipality who has been registered as a registered user of the health care database server 3 uses the vital information input transmission section 11 of a resident's health care terminal unit 1, makes access to the health care database server 3 via the Internet 6, and sends his/her vital data and/or medical questionnaire data to the health care database server 3 together with his/her user ID and password (step S3).

The user information registration verification section 31 of the health care database server 3 which received the user ID and password of the resident verifies them by referring to the user information database 35. If the authenticity of the resident as a registered user could be verified, the vital information registration section 32 which received the vital data and/or medical questionnaire data of the resident registers the data in vital information database 36. The vital data and/or medical questionnaire data of the residents of the municipality are stored with regard to each resident in the vital information database 36 (step S4).

The municipality using the database reference section 21 of the municipality's terminal unit 2 makes access to the health care database server 3 and thereby refers to the vital information database 36, classifies the matters (vital data and/or medical questionnaire data of residents) which have been stored in the vital information database 36 into matters for which a diagnosis/advice can be made by the municipality itself and matters for which a diagnosis/advice should be commissioned to an outside medical institution or health service company (step S5).

For matters for which a diagnosis/advice can be made by the municipality (“No” in step S6), the municipality makes a diagnosis, advice, etc. and inputs the diagnosis/advice to the diagnosis/advice input transmission section 22. The diagnosis/advice input transmission section 22 transmits the diagnosis/advice to the health care database server 3. If necessary, the municipality using the municipality's terminal unit 2 also obtains health-related information related to the diagnosis/advice from the health-related information database 38 of the health care database server 3 and transmits the related information to the health care database server 3 together with the diagnosis/advice (step S7).

For matters for which a diagnosis/advice should be commissioned to an outside medical institution or health service company (“Yes” in step S6), the municipality generates commission information (user ID of the resident needing a diagnosis/advice, for example) by use of the commission information generation transmission section 23 and transmits the commission information to a medical institution/health service company's terminal unit 4 of an outside medical institution or health service company to which the commission should be given (step S8).

In the medical institution/health service company's terminal unit 4 of the outside medical institution or health service company to which the commission has been given, the database reference section 41 makes access to the health care database server 3, refers to the vital information database 36, and obtains the vital data and/or medical questionnaire data of the resident corresponding to the commission information (step S9).

Subsequently, the outside medical institution or health service company makes a diagnosis, advice, etc. for the vital data and/or medical questionnaire data and inputs the diagnosis/advice to the diagnosis/advice input transmission section 42. The diagnosis/advice input transmission section 42 transmits the diagnosis/advice to the health care database server 3 via the Internet 6. If necessary, the outside medical institution or health service company using the medical institution/health service company's terminal unit 4 also obtains health-related information related to the diagnosis/advice from the health-related information database 38 of the health care database server 3 and transmits the related information to the health care database server 3 together with the diagnosis/advice (step S10).

In the health care database server 3 which received the diagnosis/advice which has been transmitted in the step S7 or S10, the diagnosis/advice registration section 33 registers and stores the diagnosis/advice for the resident in the information provision database 37. The diagnoses/advice for the residents of the municipality are stored with regard to each resident in the information provision database 37 (step S11).

The resident using the resident's health care terminal unit 1 makes access to the health care database server 3 by use of the result reference section 12, refers to the information provision database 37 and thereby obtains the diagnosis, advice, health-related information, etc. (step S12).

The provider calculates management cost of the databases periodically (once a month, for example) by use of the management cost calculation charging section 34 of the health care database server 3 and charges the municipality for the management cost by sending a bill to the municipality's terminal unit 2 via the Internet 6. The management cost is calculated according to a contract or agreement which has preliminarily been made between the municipality and the provider (based on the total number of accesses, based on the number of registered residents, etc.) (step S13).

Each of the medical institutions or health service companies also calculates diagnosis cost on each diagnosis or periodically (once a month, for example) by use of the diagnosis cost calculation charging section 43 of the medical institution/health service company's terminal unit 4 and charges the municipality for the diagnosis cost by sending a bill to the municipality's terminal unit 2 via the Internet 6 (step S14).

While the diagnosis, advice, health-related information, etc. were assumed to be obtained by a registered resident in the step S12, there are cases where another person (a relative of the resident living in a remote location, for example) intends to know health conditions of the resident. In such cases, the person can ask the municipality to register himself/herself in the user information database 35 of the health care database server 3 as a “specific user”. The registered “specific user” is allowed to make access to the health care database server 3 by use of the database reference section 51 of his/her specific user's terminal unit 5. In the health care database server 3 which received the user ID and password from the specific user, the user information registration verification section 31 verifies the user ID and password by referring to the user information database 35. If the authenticity of the specific user as a registered user could be verified, the specific user is allowed to refer to a diagnosis, advice, health-related information, etc. that have been registered in the information provision database 37 for a resident corresponding to the user ID.

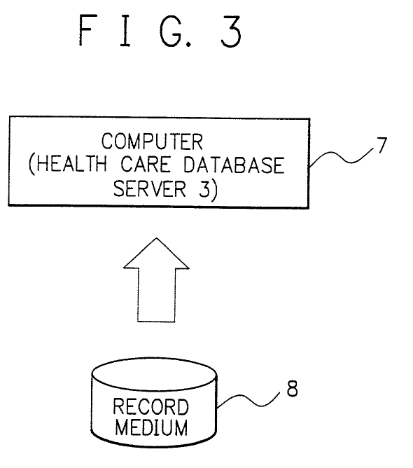

FIG. 3 is a block diagram showing an example of the implementation of the health care database server 3 of the at-home health support system of FIG. 1. The health care database server 3 shown in FIG. 3 is implemented by a computer 7 and a program stored in a record medium 8 (magnetic disc, optical disc, semiconductor memory, etc.). The program stored in the record medium 8 is read out by the computer 7 and thereby the computer 7 operates as the health care database server 3 which has been explained above.

As set forth hereinabove, in the system, method, database server and record medium for at-home health support in accordance with the present invention, the management of health care database server 3 for residents of a municipality is totally commissioned to an outside company (provider etc.), and vital data, diagnoses and advice are communicated between the health care database server 3 and terminal units (1, 2, 4) via the Internet 6. Therefore, the municipality is allowed to operate the at-home health support system without having its own server and with a small capital investment. Costs for the operation, management, maintenance, etc. of the system can be reduced in comparison with conventional cases where the municipality owns its own server for the at-home health support.

Further, even when there is no appropriate medical institution in the municipality and help from a medical institution outside the municipality is necessary, the medical institution outside the municipality can easily make access to the health care database server 3 via the Internet 6 without the need of exclusive lines.

Further, relatives etc. of the residents of the municipality who have been registered as the “specific users” are allowed to make access to the health care database server 3 from outside the municipality via the Internet 6 using the specific user's terminal units 5 and thereby refer to health conditions etc. of the residents related thereto.

Incidentally, while the calculation/charging of the database management cost and the diagnosis cost were executed by the management cost calculation charging section 34 of the health care database server 3 and the diagnosis cost calculation charging section 43 of the medical institution/health service company's terminal unit 4 respectively in the above embodiment, the calculation/charging of the costs can be conducted in various ways. For example, in extreme cases, the calculation of both the database management cost and the diagnosis cost can also be done manually by the staff of the municipality. However, cost and efficiency can be improved by employing automatic calculation by the management cost calculation charging section 34 and the diagnosis cost calculation charging section 43.

While the present invention has been described with reference to the particular illustrative embodiments, it is not to be restricted by those embodiments but only by the appended claims. It is to be appreciated that those skilled in the art can change or modify the embodiments without departing from the scope and spirit of the present invention.

What is claimed is:

1. An at-home health support system in which a health care database for residents of a municipality is constructed in a server of a company that is connected to the Internet and the municipality commissions the company to manage the health care database, comprising:
vital information registration means which are provided to the residents for sending vital data and/or medical questionnaire data of the resident to the server via the Internet and thereby registering the data of the resident in the health care database of the server;
a database reference means which is provided to the municipality for making access to the server via the Internet and thereby referring to the vital data and/or medical questionnaire data of the residents which have been registered in the health care database of the server;
a first diagnosis/advice registration means which is provided to the municipality for sending a diagnosis and/or advice made by the municipality for the vital data and/or medical questionnaire data of a resident to the server via the Internet and thereby registering the diagnosis/advice for the resident in the health care database of the server;
a diagnosis/advice commissioning means which is provided to the municipality for making access to a terminal unit of an outside medical institution or health service company via the Internet and thereby commissioning the outside medical institution or health service company to make a diagnosis and/or advice for the vital data and/or medical questionnaire data of a resident when the diagnosis/advice for the resident can not be made by the municipality;
a second diagnosis/advice registration means which is provided to the outside medical institution or health service company for sending a diagnosis and/or advice made by the outside medical institution or health service company under the commission from the municipality for the vital data and/or medical questionnaire data of the resident to the server via the Internet and thereby registering the diagnosis/advice for the resident in the health care database of the server; and
diagnosis/advice reference means which are provided to each resident for making access to the server via the Internet and thereby referring to the diagnosis/advice which has been registered in the health care database of the server.

2. An at-home health support system as claimed in claim 1, further comprising:
a management cost calculation charging means which is provided to the server for calculating management cost of the health care database and charging the municipality for the management cost via the Internet; and
a diagnosis cost calculation charging means which is provided to the outside medical institution or health service company for calculating diagnosis cost for the diagnoses/advice made by the outside medical institution or health service company under the commission from the municipality and charging the municipality for the diagnosis cost via the Internet.

3. An at-home health support system as claimed in claim 1, wherein the server includes a user verification means for checking whether or not each user making access to the server via the Internet is a registered user.

4. An at-home health support system as claimed in claim 1, further comprising second diagnosis/advice reference means which are provided to registered specific users located outside the municipality for making access to the server via the Internet and thereby letting the specific user refer to the diagnosis/advice which has been registered in the health care database of the server for a resident related to the specific user.

5. An at-home health support system as claimed in claim 4, wherein the specific users include relatives of residents of the municipality.

6. An at-home health support system as claimed in claim 1, wherein the company to which the municipality commissions the management of the health care database is an ISP (Internet Service Provider).

7. An at-home health support system in which a health care database for residents of a municipality is constructed in a server of a company that is connected to the Internet and the municipality commissions the company to manage the health care database, comprising: the server; resident's health care terminal units which are provided to residents of the municipality for making access to the Internet; a municipality's terminal unit which is provided to the municipality for making access to the Internet; and a medical institution/ health service company's terminal unit which is provided to an outside medical institution or health service company for making access to the Internet, wherein:
the resident's health care terminal unit includes:
a vital information input transmission means for letting a resident input his/her vital data and/or medical questionnaire data and transmitting the data to the server via the Internet together with a user ID and a password so that the data of the resident will be registered in the health care database of the server; and
a result reference means for making access to the server via the Internet and thereby referring to a diagnosis/advice which has been made for the vital data and/or medical questionnaire data and registered in the health care database of the server, and
the municipality's terminal unit includes:
a database reference means for making access to the server via the Internet and thereby referring to the vital data and/or medical questionnaire data of the residents which have been registered in the health care database of the server;
a first diagnosis/advice input transmission means for letting the municipality input a diagnosis and/or advice for the vital data and/or medical questionnaire data of a resident and transmitting the diagnosis/advice to the server via the Internet so that the diagnosis/advice for the resident will be registered in the health care database of the server; and
a commission information generation transmission means for generating commission information when the diagnosis/advice for a resident is commissioned to an outside medical institution or health service company and transmitting the commission information to a terminal unit of the outside medical institution or health service company via the Internet, and
the server includes:
a user information database in which resident information of each resident such as name, user ID, password, date of birth and sex and password information concerning the correspondence between each password and data/database that can be referred to by use of the password are registered and stored;
a vital information database in which the vital data, medical questionnaire data, etc. of each resident are registered and stored;
an information provision database in which the diagnosis, advice, etc. for each resident are registered and stored;
a user information registration verification means for registering user information such as the resident information and the password information in the user information database and checking whether or not each user making access to the server via the Internet is a registered user by use of the user information registered in the user information database;
a vital information registration means for registering the vital data and/or medical questionnaire data supplied from the resident's health care terminal units in the vital information database with regard to each resident; and
a diagnosis/advice registration means for registering the diagnosis/advice supplied from the municipality's terminal unit or the medical institution/health service company's terminal unit in the information provision database with regard to each resident, and
the medical institution/health service company's terminal unit includes:
a database reference means for making access to the server via the Internet and thereby referring to the vital data and/or medical questionnaire data of the residents which have been registered in the health care database of the server; and
a second diagnosis/advice input transmission means for letting the outside medical institution or health service company input a diagnosis and/or advice for the vital data and/or medical questionnaire data of a resident and transmitting the diagnosis/advice to the server via the Internet so that the diagnosis/advice for the resident will be registered in the health care database of the server.

8. An at-home health support system as claimed in claim 7, wherein:
the server further includes a management cost calculation charging means for calculating management cost of the health care database and charging the municipality for the management cost by sending a bill to the municipality's terminal unit via the Internet, and the medical institution/health service company's terminal unit further includes a diagnosis cost calculation charging means for calculating diagnosis cost for the diagnoses/advice made by the outside medical institution or health service company under the commission from the municipality and charging the municipality for the diagnosis cost by sending a bill to the municipality's terminal unit via the Internet.

9. An at-home health support system as claimed in claim 7, further comprising specific user's terminal units which are provided to registered specific users located outside the municipality for making access to the server via the Internet and thereby letting the specific user refer to the diagnosis/advice which has been registered in the health care database of the server with respect to a resident related to the specific user.

10. An at-home health support system as claimed in claim 9, wherein the specific users include relatives of residents of the municipality.

11. An at-home health support system as claimed in claim 9, wherein the user information registration verification means of the server also registers information concerning the correspondence between user IDs of the residents and passwords of the registered specific users in the user information database so that each registered specific user using a password will be allowed to refer to data of one or more residents having user IDs corresponding to the password.

12. An at-home health support system as claimed in claim 7, wherein:
the server further includes a health-related information database which preliminarily stores a variety of prepared contents concerning diet regimens, exercise regimens, rehabilitation programs, etc., and
when the diagnosis/advice is transmitted by the first diagnosis/advice input transmission means of the municipality's terminal unit or the second diagnosis/advice input transmission means of the medical institution/health service company's terminal unit to the server, health-related information related to the diagnosis/advice is obtained from the health-related information database and the health-related information is transmitted to the server together with the diagnosis/advice, and
the diagnosis/advice registration means of the server registers the health-related information in the information provision database together with the diagnosis/advice, and
the result reference means of the resident's health care terminal unit refers to the health-related information together with the diagnosis/advice.

13. An at-home health support system as claimed in claim 7, wherein the company to which the municipality commissions the management of the health care database is an ISP (Internet Service Provider).

14. A health care database server of a system including a municipality's terminal unit connected to the Internet, resident's health care terminal units connected to the Internet, a medical institution/ health service company's terminal unit connected to the Internet, and the health care database server connected to the Internet and owned by a company to which the municipality commissions the management of the health care database server, comprising:
a user information database in which resident information of each resident of the municipality such as name, user ID, password, date of birth and sex and password information concerning the correspondence between each password and data/database that can be referred to by use of the password are registered and stored;
a vital information database in which vital data, medical questionnaire data, etc. of each resident which are sent from the resident's health care terminal units are registered and stored;
an information provision database in which diagnosis, advice, etc. for each resident which are sent from the municipality's terminal unit or the medical institution/health service company's terminal unit are registered and stored;
a user information registration verification means for registering user information such as the resident information and the password information in the user information database, and checking whether or not each user making access to the health care database server via the Internet is a registered user by use of the user information registered in the user information database when access from the municipality's terminal unit, the resident's health care terminal unit or the medical institution/health service company's terminal unit occurred;
a vital information registration means for registering the vital data and/or medical questionnaire data supplied from the resident's health care terminal units via the Internet in the vital information database with regard to each resident so as to be referred to by the municipality's terminal unit or the medical institution/health service company's terminal unit; and
a diagnosis/advice registration means for registering the diagnosis/advice supplied from the municipality's terminal unit or the medical institution/health service company's terminal unit via the Internet in the information provision database with regard to each resident so as to be referred to by the resident's health care terminal unit.

15. A health care database server as claimed in claim 14, further comprising a management cost calculation charging means for calculating management cost of the health care database server and charging the municipality for the management cost by sending a bill to the municipality's terminal unit via the Internet.

16. An at-home health support method in which a health care database for residents of a municipality is constructed in a server of a company that is connected to the Internet and the municipality commissions the company to manage the health care database, comprising the steps of:
a vital information registration step in which a resident sends his/her vital data and/or medical questionnaire data to the server via the Internet by use of a resident's health care terminal unit and thereby the data of the resident is registered in the health care database of the server;
a database reference step in which the municipality makes access to the server via the Internet by use of a municipality's terminal unit and thereby refers to the vital data and/or medical questionnaire data of the resident which have been registered in the health care database of the server;
a first diagnosis/advice registration step in which the municipality makes a diagnosis and/or advice for the vital data and/or medical questionnaire data of the resident if possible and sends the diagnosis/advice from the municipality's terminal unit to the server via the Internet and thereby the diagnosis/advice for the resident is registered in the health care database of the server;
a diagnosis/advice commissioning step in which the municipality makes access to a medical institution/health service company's terminal unit of an outside medical institution or health service company via the Internet by use of the municipality's terminal unit and thereby commissions the outside medical institution or health service company to make a diagnosis and/or advice for the vital data and/or medical questionnaire data of the resident if the diagnosis/advice for the resident should be commissioned to the outside medical institution or health service company;
a second diagnosis/advice registration step in which the outside medical institution or health service company makes a diagnosis and/or advice for the vital data and/or medical questionnaire data of the resident under the commission from the municipality and sends the diagnosis/advice from the medical institution/health service company's terminal unit to the server via the Internet and thereby the diagnosis/advice for the resident is registered in the health care database of the server; and
a diagnosis/advice reference step in which the resident makes access to the server via the Internet by use of the resident's health care terminal unit and thereby refers to the diagnosis/advice which has been registered in the health care database of the server.

17. An at-home health support method as claimed in claim 16, further comprising:
a management cost calculation charging step in which management cost of the health care database is calculated by the server and the management cost is charged to the municipality by sending a bill to the municipality's terminal unit via the Internet; and
a diagnosis cost calculation charging step in which diagnosis cost for the diagnoses/advice made by the outside medical institution or health service company under the commission from the municipality is calculated by the medical institution/health service company's terminal unit and the diagnosis cost is charged to the municipality by sending a bill to the municipality's terminal unit via the Internet.

18. An at-home health support method as claimed in claim 16, wherein the outside medical institution or health service company which received the commission from the municipality in the diagnosis/advice commissioning step makes access to the server via the Internet by use of the medical institution/health service company's terminal unit and thereby refers to the vital data and/or medical questionnaire data of the resident which have been registered in the health care database of the server.

19. An at-home health support method as claimed in claim 16, further comprising a user verification step in which the authenticity of a user making access to the server via the Internet as a registered user is checked.

20. An at-home health support method as claimed in claim 16, further comprising a second diagnosis/advice reference step in which a registered specific user located outside the municipality makes access to the server via the Internet by use of a specific user's terminal unit and thereby refers to the diagnosis/advice which has been registered in the health care database of the server for a resident related to the specific user.

21. An at-home health support method as claimed in claim 20, wherein the specific users include relatives of residents of the municipality.

22. An at-home health support system as claimed in claim 16, wherein the company to which the municipality commissions the management of the health care database is an ISP (Internet Service Provider).

23. A computer-readable record medium storing a program for instructing a computer etc. to operate as a health care database server of a system including a municipality's terminal unit connected to the Internet, resident's health care terminal units connected to the Internet, a medical institution/health service company's terminal unit connected to the Internet, and the health care database server connected to the Internet and owned by a company to which the municipality commissions the management of the health care database server, wherein the health care database server implemented by the computer etc. and the program comprises:
a user information database in which resident information of each resident of the municipality such as name, user ID, password, date of birth and sex and password information concerning the correspondence between each password and data/database that can be referred to by use of the password are registered and stored;
a vital information database in which vital data, medical questionnaire data, etc. of each resident which are sent from the resident's health care terminal units are registered and stored;
an information provision database in which diagnosis, advice, etc. for each resident which are sent from the municipality's terminal unit or the medical institution/health service company's terminal unit are registered and stored;
a user information registration verification means for registering user information such as the resident information and the password information in the user information database, and checking whether or not each user making access to the health care database server via the Internet is a registered user by use of the user information registered in the user information database when access from the municipality's terminal unit, the resident's health care terminal unit or the medical institution/health service company's terminal unit occurred;
a vital information registration means for registering the vital data and/or medical questionnaire data supplied from the resident's health care terminal units via the Internet in the vital information database with regard to each resident so as to be referred to by the municipality's terminal unit or the medical institution/health service company's terminal unit; and
a diagnosis/advice registration means for registering the diagnosis/advice supplied from the municipality's terminal unit or the medical institution/health service company's terminal unit via the Internet in the information provision database with regard to each resident so as to be referred to by the resident's health care terminal unit.

24. A computer-readable record medium as claimed in claim 23, wherein the health care database server implemented by the computer etc. and the program further comprises a management cost calculation charging means for calculating management cost of the health care database server and charging the municipality for the management cost by sending a bill to the municipality's terminal unit via the Internet.
